(19) United States
(12) Patent Application Publication
(10) Pub. No.: US 2019/0358209 A1
(43) Pub. Date: Nov. 28, 2019

(54) DRUG COMPOSITIONS

(71) Applicant: Adamis Pharmaceuticals Corporation, San Diego, CA (US)

(72) Inventor: Dennis J. Carlo, San Diego, CA (US)

(21) Appl. No.: 16/422,527

(22) Filed: May 24, 2019

Related U.S. Application Data
(60) Provisional application No. 62/676,750, filed on May 25, 2018.

(51) Int. Cl. A61K 31/439; A61K 31/737; A61K 31/195

(52) U.S. Cl. CPC A61K 31/439; A61K 31/195; A61K 31/737

(57) ABSTRACT

Described herein are pharmaceutical compositions for treating pain.

### CROSS REFERENCE TO RELATED APPLICATIONS

This application claims the benefit of U.S. provisional patent application No. 62/676,750 filed May 25, 2018, the entire disclosure of which is incorporated herein by reference.

### FIELD

Described herein are compositions including combination therapy for treating pain.

### SUMMARY

The Institute of Medicine estimates more than 100 million Americans suffer from persistent or chronic pain with 10% experiencing chronic debilitating pain. Common pain treatments include opioids which are associated with significant morbidity due to opioid use disorders. Alternative safe treatments are warranted.

Described herein are pharmaceutical compositions useful for treating and/or preventing pain in mammals. Mammals can include, but are not limited to humans, horses, camels, dogs, cats, cows, bears, rodents, sheep, goats, pigs and the like. In some embodiments, the compositions are useful in humans. In other embodiments, the compositions described herein can be considered veterinary compositions. In some embodiments, the compositions can include a synergistic combination of drugs and/or have an additive drug effect and can be termed combination therapy. In some embodiments, the combination therapy can include an opioid antagonist, a heparin-like compound, and/or gabapentin.

In one embodiment, the compositions can include, combined, an opioid antagonist and a heparin-like compound. In another embodiment, the compositions can include, combined, an opioid antagonist and gabapentin. In still other embodiments, the compositions can include, combined, an opioid antagonist, a heparin-like compound, and gabapentin.

In some embodiments, the opioid antagonist can be naltrexone, a derivative thereof, a prodrug thereof, or a salt form thereof.

In some embodiments, the heparin-like compound can be pentosan polysulfate sodium, a derivative thereof, a prodrug thereof, or a salt form thereof.

In some embodiment, the compositions include gabapentin.

In some embodiments, combining naltrexone with pentosan polysulfate sodium can provide a surprising synergetic effect that can reduce and/or prevent pain. In other embodiments, combining naltrexone with gabapentin can provide a surprising synergetic effect that can reduce and/or prevent pain. In other embodiments, the combinations can reduce the amount of each component needed to achieve a similar or better result when compared to the single component.

In some embodiments, combining naltrexone with pentosan polysulfate sodium can provide an additive effect that can reduce and/or prevent pain.

In some embodiments, combining naltrexone with gabapentin can provide an additive effect that can reduce and/or prevent pain.

In some embodiments, the compositions are formulated as a non-solid or liquid for injection. In other embodiments, the compositions are formulated for oral administration. The non-solid can be a liquid. In other embodiments, the liquid can be formulated for either local injection or systemic injection.

In some embodiments, one or more of the active agents/drugs can be administered orally. In some embodiments, all the active agents/drugs can be administered orally.

Methods of treating pain are also described. The methods can comprise administering a composition including an opioid antagonist, a heparin-like compound, and/or gabapentin to a mammal having or experiencing pain.

### DETAILED DESCRIPTION

Described herein are pharmaceutical compositions useful for treating and/or preventing pain in mammals. In some embodiments, the pain can be acute pain, chronic pain, neuropathic pain, postoperative pain, bladder pain, muscle pain, wound pain, ligament pain, joint pain, inflammatory pain, surgical pain, or a combination thereof. In some embodiments, the different types of pain enumerated can be interrelated. In one embodiment, the bladder pain is interstitial cystitis. In other embodiments, the pain is associated with fibromyalgia, Crohn's, and/or multiple sclerosis.

Mammals can include, but are not limited to, humans, horses, camels, dogs, cats, cows, bears, rodents, sheep, goats, pigs and the like.

Treatment of pain can involve pharmaceuticals that have many and/or severe side effects. Prior to discovery of the present compositions, treatment of pain may require a patient to take large doses of drugs or small does of high powered drugs. In some cases, drugs for treating pain can have psychological effects. In some cases patients take drugs having very severe/serious side effects; in some cases, these drugs are taken in very large doses. Generally, doses can increase as a disorder progresses. In some circumstances, no cure exists and the therapies endure indefinitely. Thus, the longer a mammal is subjected to treatment for pain or an underlying disorder with a particular drug(s), the more side effects the mammal may encounter.

The present combinations can produce synergistic and/or additive effects in reducing at least one symptom associated with pain. In some embodiments, the present combinations can eliminate pain. Consequently, in some embodiments, a reduced, e.g., considerably, dose of therapeutic compounds can be given for an equivalent effect for each individual therapeutic compound. In other embodiments, an equivalent amount of each therapeutic compound, when compared to single compound treatment, can be given to achieve a larger and/or more rapid response. In some embodiments, the compositions can reduce side-effects and drug burden.

As used herein, the term “pharmaceutical composition” refers to a therapeutically effective concentration of the drugs and other ingredients described herein. As used herein, the term “pharmaceutically acceptable” refers to compositions that do not produce an adverse, allergic, or other untoward or unwanted reaction when administered to a mammal.

In some embodiments, the compositions can include an opioid antagonist, a heparin-like compound, and/or gabapentin.

In one embodiment, the compositions can include, combined, an opioid antagonist and a heparin-like compound. In another embodiment, the compositions can include, combined, an opioid antagonist and gabapentin. In still other embodiments, the compositions can include, combined, an opioid antagonist, a heparin-like compound, and gabapentin.

In some embodiments, the opioid antagonist can be naltrexone, a derivative thereof, a prodrug thereof, or a salt form thereof. Naltrexone is an opioid antagonist that binds and inactivates opioid receptors. In some embodiments herein, naltrexone is delivered at a low dose. Low dose naloxone (LDN) can have a paradoxical anti-inflammatory effect. This effect can reduce pain, e.g., chronic pain and pain symptoms.

In some embodiments, naltrexone has a structure

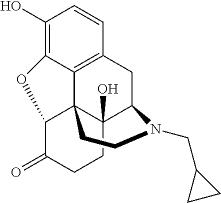

and can include pharmaceutically acceptable salts thereof.

In some embodiments, the opioid antagonist can be administered in the composition at a concentration of about 50 mg to about 100 mg, about 50 mg to about 200 mg, about 75 mg to about 100 mg, about 100 mg to about 200 mg, or about 50 mg to about 75 mg. In some embodiments, the opioid antagonist can be administered in the composition at a low dose of about 0.1 mg to about 10 mg, about 0.5 mg to about 10 mg, about 0.1 mg to about 5 mg, about 0.5 mg to about 5 mg, about 0.1 mg to about 20 mg, about 1 mg to about 10 mg, or about 1 mg to about 20 mg.

In some embodiments, the opioid antagonist can be administered once a day, twice a day, three times a day, four times a day, five times a day, six times a day or more. In some embodiments, the opioid antagonist amounts can be delivered in more than one administration.

In some embodiments, naltrexone can be injected directly to the site of treatment. In one embodiment, when treating a bladder disorder such as, but not limited to interstitial cystitis, naltrexone can be injected directly to the bladder. In such a direct injection treatment, the opioid antagonist can be injected with the heparin-like compound, gabapentin, or both, or injected or administered separately to the same location or another location entirely.

In some embodiments, naltrexone can be administered orally. In some embodiments, the opioid antagonist can be orally administered with the heparin-like compound, gabapentin, or both, or administered separately.

In some embodiments, naltrexone can be inhaled. This inhalation can be with the heparin-like compound, gabapentin, or both, or administered separately.

In some embodiments, the opioid antagonist can be injected directly to the site of treatment. In one embodiment, when treating a bladder disorder such as, but not limited to interstitial cystitis, the opioid antagonist can be injected directly to the bladder. In such a direct injection treatment, the opioid antagonist can be injected with the heparin-like compound, gabapentin, or both, or injected or administered separately to the same location or another location entirely.

In some embodiments, the opioid antagonist can be administered orally. In some embodiments, the opioid antagonist can be orally administered with the heparin-like compound, gabapentin, or both, or administered separately.

In some embodiments, the opioid antagonist can be inhaled. This inhalation can be with the heparin-like compound, gabapentin, or both, or administered separately.

In some embodiments, the opioid antagonist can be included in the composition at most about 35% (w/v), at most about 40% (w/v), about 15% (w/v), about 20% (w/v), about 25% (w/v), about 30% (w/v), about 35% (w/v), about 40% (w/v), about 45% (w/v), about 50% (w/v), between about 20% and about 40% (w/v), or between about 25% and about 35% (w/v).

In some embodiments, naltrexone can be administered in the composition at a concentration of about 50 mg to about 100 mg, about 50 mg to about 200 mg, about 75 mg to about 100 mg, about 100 mg to about 200 mg, or about 50 mg to about 75 mg. In some embodiments, naltrexone can be administered in the composition at a low dose of about 0.1 mg to about 10 mg, about 0.5 mg to about 10 mg, about 0.1 mg to about 5 mg, about 0.5 mg to about 5 mg, about 0.1 mg to about 20 mg, about 1 mg to about 10 mg, or about 1 mg to about 20 mg.

In some embodiments, naltrexone can be administered once a day, twice a day, three times a day, four times a day, five times a day, six times a day or more. In some embodiments, naltrexone amounts can be delivered in more than one administration.

In some embodiments, the heparin-like compound can be administered in the composition at a concentration of about 50 mg to about 100 mg, about 50 mg to about 200 mg, about 75 mg to about 100 mg, about 100 mg to about 200 mg, or about 50 mg to about 75 mg.

In some embodiments, the heparin-like compound can be administered once a day, twice a day, three times a day, four times a day, five times a day, six times a day or more. In some embodiments, the heparin-like compound amounts can be delivered in more than one administration.

In some embodiments, the heparin-like compound can be injected directly to the site of treatment. In one embodiment, when treating a bladder disorder such as, but not limited to interstitial cystitis, the heparin-like compound can be injected directly to the bladder. In such a direct injection treatment, the opioid antagonist can be injected with the heparin-like compound, gabapentin, or both, or injected or administered separately to the same location or another location entirely.

In some embodiments, the heparin-like compound can be administered orally. In some embodiments, the heparin-like compound can be orally administered with the opioid antagonist, gabapentin, or both, or administered separately.

In some embodiments, the heparin-like compound can be inhaled. This inhalation can be with the opioid antagonist, gabapentin, or both, or administered separately.

In some embodiments, the heparin-like compound can be included in the composition at most about 30% (w/v), at most about 25% (w/v), about 10% (w/v), about 15% (w/v), about 20% (w/v), about 21% (w/v), about 22% (w/v), about 23% (w/v), about 25% (w/v), about 30% (w/v), about 35% (w/v), about 40% (w/v),between about 20% and about 25% (w/v), or between about 15% and about 30% (w/v).

In some embodiments, the heparin-like compound can be pentosan polysulfate sodium, a derivative thereof, a prodrug thereof, or a salt form thereof.

In some embodiments, pentosan polysulfate sodium has a structure

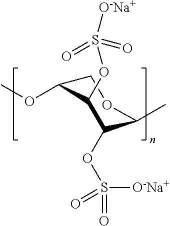

wherein n is 1 to 1,000.

Pentosan polysulfate sodium is a semi-synthetically produced heparin-like macromolecular carbohydrate derivative, which chemically and structurally resembles glycosaminoglycans. Pentosan polysulfate sodium can be referred to as a low molecular weight heparin-like compound. It can have anticoagulant and fibrinolytic effects.

In some embodiments, the pentosan polysulfate does not include sodium. Pentosan polysulfate can include K, Ca, Mg, and/or another suitable cation. Pharmaceutically acceptable salts of pentosan polysulfate are also included.

In some embodiments, pentosan polysulfate sodium can be administered in the composition at a concentration of about 50 mg to about 100 mg, about 50 mg to about 200 mg, about 75 mg to about 100 mg, about 100 mg to about 200 mg, or about 50 mg to about 75 mg.

In some embodiments, pentosan polysulfate sodium can be administered once a day, twice a day, three times a day, four times a day, five times a day, six times a day or more. In some embodiments, pentosan polysulfate sodium amounts can be delivered in more than one administration.

In some embodiments, the pentosan polysulfate sodium can be injected directly to the site of treatment. In one embodiment, when treating a bladder disorder such as, but not limited to interstitial cystitis, the pentosan polysulfate sodium can be injected directly to the bladder. In such a direct injection treatment, the pentosan polysulfate sodium can be injected with the opioid antagonist, gabapentin, or both, or injected or administered separately to the same location or another location entirely.

In some embodiments, the pentosan polysulfate sodium can be administered orally. In some embodiments, the pentosan polysulfate sodium can be orally administered with the opioid antagonist, gabapentin, or both, or administered separately.

In some embodiments, the pentosan polysulfate sodium can be inhaled. This inhalation can be with the opioid antagonist, gabapentin, or both, or administered separately.

In some embodiments, the compositions can include gabapentin. In some embodiments, gabapentin has a structure

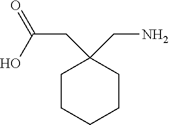 [SMILES: NCC1(CC(=O)O)CCCCC1]

and can include pharmaceutically acceptable salts thereof.

Gabapentin's mechanism of action can be through a complex synergy between increased GABA synthesis, non-NMDA receptor antagonism, and binding to the a2d subunit of voltage dependent calcium channels. Its effect on calcium channels can be by inhibiting release of excitatory neurotransmitters.

In some embodiments, gabapentin can be administered in the composition at a concentration of about 900 mg to about 1,200 mg, about 1,000 mg to about 1,500 mg, about 750 mg to about 1,000 mg, about 900 mg to about 1,200 mg, or about 900 mg to about 1,500 mg.

In some embodiments, gabapentin can be administered once a day, twice a day, three times a day, four times a day, five times a day, six times a day or more. In some embodiments, gabapentin amounts can be delivered in more than one administration.

In some embodiments, the gabapentin can be injected directly to the site of treatment. In one embodiment, when treating a bladder disorder such as, but not limited to interstitial cystitis, the gabapentin can be injected directly to the bladder. In such a direct injection treatment, the gabapentin can be injected with the opioid antagonist, heparin-like compound, or both, or injected or administered separately to the same location or another location entirely.

In some embodiments, the gabapentin can be administered orally. In some embodiments, the gabapentin can be orally administered with the opioid antagonist, heparin-like compound, or both, or administered separately.

In some embodiments, the gabapentin can be inhaled. This inhalation can be with the opioid antagonist, heparin-like compound, or both, or administered separately.

In some embodiments, the gabapentin can be included in the composition at most about 30% (w/v), at most about 25% (w/v), about 10% (w/v), about 15% (w/v), about 20% (w/v), about 21° A (w/v), about 22% (w/v), about 23% (w/v), about 25% (w/v), about 30% (w/v), about 35% (w/v), about 40% (w/v),between about 20% and about 25% (w/v), or between about 15% and about 30% (w/v).

In some embodiments, the opioid antagonist, heparin-like compound, and gabapentin can be injected in a single composition. In some embodiments, the opioid antagonist, heparin-like compound, and gabapentin can be injected in two or more compositions at about the same time.

In some embodiments, the opioid antagonist, heparin-like compound, and gabapentin can be administered orally in a single composition. In some embodiments, the opioid antagonist, heparin-like compound, and gabapentin can be administered orally in two or more compositions at about the same time.

In some embodiments, the opioid antagonist, heparin-like compound, and gabapentin can be inhaled in a single composition. In some embodiments, the opioid antagonist, heparin-like compound, and gabapentin can be inhaled in two or more compositions at about the same time.

These components, the opioid antagonist, heparin-like compound, and gabapentin each have a different mechanism(s) of action. Thus, the combination of these components has can result in synergy for analgesia and pain control and/or management. Further, in some embodiments, the combinations are well tolerated with limited side effects.

In still other embodiments, the combinations can be administered without a potential for dependence.

In some embodiments, each of the opioid antagonist, heparin-like compound, and gabapentin can be delivered in particle weights per day. Weights can include between about 10 mg and about 1,000 mg, between about 10 mg and about 100 mg, between about 10 mg and about 500 mg, between about 10 mg and about 50 mg, between about 10 mg and about 400 mg, between about 10 mg and about 300 mg, between about 10 mg and about 200 mg, between about 100 mg and about 400 mg, between about 100 mg and about 500 mg, or between about 1 mg and about 1,000 mg per day.

In some embodiments, the opioid antagonist can be delivered at a dose of between about 10 mg and about 500 mg, between about 10 mg and about 200 mg, between about 10 mg and about 100 mg, between about 10 mg and about 50 mg, between about 10 mg and about 250 mg, between about 10 mg and about 300 mg, between about 50 mg and about 100 mg, between about 500 mg and about 200 mg, or between about 50 mg and about 150 mg per day.

In other embodiments, the opioid antagonist can be delivered at a low dose of between about 0.1 mg and about 10 mg, between about 0.1 mg and about 20 mg, between about 0.1 mg and about 50 mg, between about 0.5 mg and about 10 mg, between about 0.05 mg and about 10 mg, between about 0.05 mg and about 20 mg, or between about 0.1 mg and about 5 mg per day. In some embodiments, low doses of the opioid antagonist can have at least an anti-inflammatory effect.

The drug combination or combination therapy may be made into a liquid formulation. Liquid formulations suitable for enteral or parenteral administration include, without limitation, solutions, syrups, elixirs, dispersions, emulsions, and suspensions. The drug combination or combination therapy disclosed herein intended for such administration may be prepared according to any method known in the art for the manufacture of pharmaceutical compositions. In such liquid dosage forms, the drug combination disclosed herein may be admixed with (a) suitable aqueous and non-aqueous carriers, (b) diluents, (c) solvents, such as, e.g., water, ethanol, propylene glycol, polyethyleneglycol, glycerol, vegetable oils, such as, e.g., rapeseed oil and olive oil, and injectable organic esters such as ethyl oleate; and/or fluidity agents, such as, e.g., surfactants or coating agents like lecithin. In the case of dispersions and suspensions, fluidity can also be controlled by maintaining a particular particle size.

The drug combination or combination therapy disclosed herein may be made into a semi-solid formulation. Semi-solid formulations can be made for enteral or topical administration. Semi-solid formulations suitable for enteral administration include, without limitation, pastes, and gels. Semi-solid formulations suitable for topical or oral administration include, without limitation, ointments, creams, salves, pastes, and gels. The drug combination or combination therapy disclosed herein intended for such administration may be prepared according to any method known in the art for the manufacture of pharmaceutical compositions.

Liquid and/or semi-solid formulations may be formulated with sweetening agents, for example glycerol, propylene glycol, sorbitol or sucrose. Such formulations may also contain one or more demulcent, preservative, flavoring agent, and/or coloring agent.

Liquid and/or semi-solid suspensions may be formulated by suspending the drug combination or components of the combination in an admixture with suitable excipients. Suitable excipients can be suspending agents, such as sodium carboxymethyl cellulose, methylcellulose, hydroxypropylmethylcellulose, sodium alginate, pectin, polyvinyl pyrrolidone, polyvinyl alcohol, natural gum, agar, gum tragacanth and gum acacia.

Oily suspensions may be formulated by suspending the drug combination in an admixture with (a) vegetable oils including almond oil, arachis oil, avocado oil, canola oil, castor oil, coconut oil, corn oil, cottonseed oil, grape seed oil, hazelnut oil, hemp oil, linseed oil, olive oil, palm oil, peanut oil, rapeseed oil, rice bran oil, safflower oil, sesame oil, soybean oil, soya oil, sunflower oil, walnut oil, wheat germ oil, or a combination thereof, (b) a saturated fatty acid, an unsaturated fatty acid, or a combination thereof, such as palmitic acid, stearic acid, oleic acid, linoleic acid, linolenic acid, or a combination thereof, (c) mineral oil such as liquid paraffin, (d) surfactants or detergents. The oily suspensions may contain a thickening agent such as beeswax, hard paraffin, or cetyl alcohol.

In some embodiments, the liquid or semi solid formulations can include liposomes. More specifically, liposomes can be loaded with drugs or a particular drug of a herein described combination. Liposomes include phospoholipid bilayers and can be formulated with various diameters. In some embodiments, the liposomes can be micrometer(s) in size. In other embodiments, the liposomes can be nanometer(s) in size.

In some embodiments, the liposomes can mimic cell walls and/or protect the drug that it encases. In some embodiments, liposomes can fuse with a cell wall and allow a drug(s) to be delivered into the cell.

Liposomes can be used to encapsulate the opioid antagonist. In other embodiments, the liposomes can encapsulate the heparin-like compound. In other embodiments, the liposomes can encapsulate the gabapentin. In still other embodiments, liposomes can encapsulate the opioid antagonist, the heparin-like compound, and the gabapentin in the same liposome. In still other embodiments, liposomes can encapsulate the opioid antagonist and the heparin-like compound in the same liposome. In still other embodiments, liposomes can encapsulate the opioid antagonist and the gabapentin in the same liposome. In still other embodiments, liposomes can encapsulate the heparin-like compound and the gabapentin in the same liposome.

In some embodiments, the opioid antagonist is loaded into a liposome as described herein for delivery. In other embodiments, the heparin-like compound is loaded into a liposome as described herein for delivery. In other embodiments, the gabapentin is loaded into a liposome as described herein for delivery. In still other embodiments, liposomes can encapsulate two or three of the drugs in the same liposome for delivery.

In some embodiments, the liposomes can be between about 50 nm and about 200 nm, between about 100 nm and about 500 nm, between about 500 nm and about 1,000 nm, between about 1,000 nm and about 5,000 nm, between about 1 nm and about 500 nm, between about 10 nm and about 100 nm, between about 10 nm and about 250 nm, between about 10 nm and about 1,000 nm, between about 10 nm and about 1,000 nm, or between about 10 nm and about 5,000 nm in diameter.

A composition disclosed herein may optionally include a pharmaceutically-acceptable carrier that facilitates processing of drug(s) into pharmaceutically-acceptable compositions. Such a carrier can be mixed with a drug or drugs or permitted to dilute or enclose the drug or drugs and can be a solid, semi-solid, or liquid agent. It is understood that the drug or drugs can be soluble or can be delivered as a suspension in the desired carrier or diluent. Any of a variety of pharmaceutically acceptable carriers can be used including, without limitation, aqueous media such as, e.g., water, saline, glycine, and the like; solid carriers such as, e.g., polyethylene glycol, polyethylene oxide, mannitol, lactose, starch, magnesium stearate, sodium saccharin, talcum, cellulose, glucose, sucrose, magnesium carbonate, and the like; solvents; dispersion media; coatings; isotonic and absorption delaying agents; or any other inactive ingredient. Selection of a pharmacologically acceptable carrier can depend on the mode of administration.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, flavoring agents, coloring agents, suspension agents, and the like.

Various buffers and means for adjusting pH can be used to prepare a pharmaceutical composition disclosed herein. Such buffers include, without limitation, acetate buffers, citrate buffers, phosphate buffers, neutral buffered saline, phosphate buffered saline, bicarbonate buffers, and borate buffers. It is understood that acids or bases can be used to adjust the pH of a composition as needed.

Bases can include sodium hydroxide. In one embodiment, sodium bicarbonate is used as a buffer. In another embodiment, sodium hydroxide is used to make the composition more alkaline. In some embodiments, the alkalinity of the composition can be a pH greater than about 7, greater than about 7.5, greater than about 8, greater than about 8.5, greater than about 9, greater than about 9.5, or greater than about 10.

Acids can include hydrochloric acid. In one embodiment, hydrochloric acid is used to make the composition more acidic. In some embodiments, the acididy of the composition can be a pH less than about 7, less than about 6.5, less than about 6, less than about 5.5, less than about 5, less than about 4.5, or less than about 4.

In some embodiments, a buffer(s) can be included in the composition at most about 10% (w/v), at most about 5% (w/v), about 1% (w/v), about 2% (w/v), about 3% (w/v), about 4% (w/v), about 5% (w/v), about 6% (w/v), about 7% (w/v), about 8% (w/v), about 9% (w/v), about 10% (w/v), between about 1% and about 10% (w/v), or between about 4% and about 6% (w/v).

Pharmaceutically acceptable antioxidants can include, without limitation, sodium metabisulfite, sodium thiosulfate, acetylcysteine, butylated hydroxyanisole and butylated hydroxytoluene.

If formulated for oral delivery, flavoring agents can provide a composition that smells good and/or tastes good. Mammals may need encouragement to consume the compositions described. Thus, flavoring agents may be added to stimulate appeal or naturally attract a particular mammal species. Flavoring agents can make orally administrable compositions taste like apple, orange, lemon, grape, butterscotch, cherry, blueberry, raspberry, strawberry, honey, peppermint, spearmint, cinnamon, peach, watermelon, chocolate, espresso, mango, banana, carrot, cantaloupe, guava, acai, cheese, tomato, caramel, taffy, lime, and the like. Flavor combinations can also be provided.

Preservatives can include, without limitation, sodium sulfite, sodium sulfide, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. In one embodiment, sodium sulfite is used as a preservative.

In some embodiments, a preservative(s) can be included in the composition at most about 0.2% (w/v), at most about 0.5% (w/v), about 0.05% (w/v), about 0.06% (w/v), about 0.07% (w/v), about 0.08% (w/v), about 0.09% (w/v), about 0.1% (w/v), about 0.11% (w/v), about 0.12% (w/v), about 0.13% (w/v), about 0.14% (w/v), about 0.15% (w/v), between about 0.05% and about 0.15% (w/v), or between about 0.09% and about 0.11% (w/v).

Tonicity adjustors can include, without limitation, salts such as, e.g., sodium chloride, potassium chloride, mannitol or glycerin and other pharmaceutically acceptable tonicity adjustors.

Suspension agents can include, without limitation, carboxymethyl cellulose. The suspension agent can be used to keep the drug(s) dispersed evenly throughout the composition.

In some embodiments, the suspension agent(s) can be included in the composition at most about 0.2% (w/v), at most about 0.5% (w/v), about 0.05% (w/v), about 0.1% (w/v), about 0.2% (w/v), about 0.3% (w/v), about 0.4% (w/v), about 0.5% (w/v), about 0.8% (w/v), about 1% (w/v), between about 0.1% and about 0.3% (w/v), or between about 0.1% and about 1% (w/v).

In other embodiments, the drug combination disclosed herein may be made into an inhaled formulation. Inhaled formulations suitable for enteral or parenteral administration include, without limitation, aerosols, and dry powders. The drug combination disclosed herein intended for such administration may be prepared according to any method known in the art for the inhalable manufacture of pharmaceutical compositions.

The drug combination may be made into a solid formulation. Solid formulations suitable for enteral or parenteral administration include, without limitation, capsules, tablets, pills, troches, lozenges, powders and granules suitable for inhalation or for reconstitution into sterile injectable solutions or dispersions. The drug combination intended for such administration may be prepared according to any method known to the art for the manufacture of pharmaceutical compositions. In such solid dosage forms, the drug combination may be admixed with (a) at least one inert customary excipient (or carrier), such as, e.g., sodium citrate or dicalcium phosphate or (b) fillers or extenders, as for example, starch, lactose, sucrose, glucose, mannitol, isomalt, and silicic acid, (c) binders, such as, e.g., carboxymethylcellulose, alignates, gelatin, polyvinylpyrrolidone, sucrose and acacia, (d) humectants, such as, e.g., glycerol, (e) disintegrating agents, such as, e.g., agar-agar, calcium carbonate, corn starch, potato starch, tapioca starch, alginic acid, certain complex silicates and sodium carbonate, (f) solution retarders, such as, e.g., paraffin, (g) absorption accelerators, such as, e.g., quaternary ammonium compounds, (h) wetting agents, such as, e.g., cetyl alcohol and glycerol monostearate, (i) adsorbents, such as, e.g., kaolin and bentonite, (j) lubricants, such as, e.g., talc, stearic acid, calcium stearate, magnesium stearate, solid polyethylene glycols, sodium lauryl sulfate or mixtures thereof, and (k) buffering agents. The tablets may be uncoated or they may be coated by known techniques to delay disintegration and absorption in the gastrointestinal tract and thereby provide a sustained action over a longer period.

Combining an opioid antagonist with a heparin-like compound and/or gabapentin can provide a surprising synergistic effect that can reduce symptoms associated with pain. In other embodiments, combining opioid antagonist with a heparin-like compound and/or gabapentin can provide a surprising synergistic effect that can prevent symptoms associated with pain.

The compositions disclosed herein may be formulated for either local or systemic delivery. In some embodiments, one components may be formulated for local delivery and another for systemic delivery.

In some embodiments, a composition disclosed herein can be provided for injection, e.g., local injection. In other embodiments, the compositions can be injected systemically. In other embodiments, combinations of injections paradigms can be utilized.

In some embodiments, the pentosan polysulfate sodium can be injected locally while the opiod antagonist and/or gabapentin are delivered systemically. In other embodiments, the pentosan polysulfate sodium and the opiod antagonist and/or gabapentin can all be delivered systemically. In still other embodiments, the pentosan polysulfate sodium and the opiod antagonist and/or gabapentin can all be injected locally. In some embodiments, the pentosan polysulfate sodium and the opiod antagonist and/or gabapentin can be injected together in an single injection or can be injected separately.

The compositions described herein can be administered at amounts of about 10 cc, about 15 cc, about 20 cc, about 25 cc, about 30 cc, about 35 cc, about 40 cc, about 45 cc, about 50 cc, about 55 cc, about 60 cc, about 65 cc, about 70 cc, about 75 cc, about 80 cc, about 85 cc, about 90 cc, about 95 cc, about 100 cc, at least about 10 cc, at least about 20 cc, between about 20 cc and about 50 cc, between about 20 cc and about 30 cc, between about 25 cc and about 35 cc, between about 30 cc and about 35 cc, or between about 25 cc and about 30 cc per administration.

Administration can be any interval that results in a therapeutic response. In some embodiments, administration can be one, two, three, four, five, or more times per day. In other embodiments, administration can be every other day, every third day, every fourth day, every fifth day, every sixth day, once per week, twice per month, monthly, and the like. In one embodiment, administration is once per day.

In one embodiment, the compositions can include, combined, an opioid antagonist and a heparin-like compound. In another embodiment, the compositions can include, combined, an opioid antagonist and gabapentin. In still other embodiments, the compositions can include, combined, an opioid antagonist, a heparin-like compound, and gabapentin.

Methods of forming a composition including an opioid antagonist, a heparin-like compound, and/or gabapentin are also described. The methods can comprise mixing a combination including the opioid antagonist and the heparin-like compound in a composition for administration. In other embodiments, the methods can comprise mixing a combination including the opioid antagonist and gabapentin in a composition for administration. In still other embodiments, the methods can comprise mixing a combination including the opioid antagonist, the heparin-like compound, and gabapentin in a composition for administration.

In other embodiments, methods can comprise mixing a combination including naltrexone and gabapentin in a composition for administration. In other embodiments, methods can comprise mixing a combination including naltrexone and pentosan polysulfate sodium a composition for administration. In still other embodiments, the methods can comprise mixing a combination including naltrexone, pentosan polysulfate sodium, and gabapentin in a composition for administration.

In some embodiments, the mixed composition can also optionally include a preservative as described herein.

Some embodiments provide a kit including a syringe pre-filled with a composition including an opioid antagonist and a heparin-like compound, and instructions for use in a unifying container.

Some embodiments provide a kit including a syringe pre-filled with a composition including an opioid antagonist and gabapentin, and instructions for use in a unifying container.

Some embodiments provide a kit including a syringe pre-filled with a composition including an opioid antagonist, a heparin-like compound, and gabapentin and, instructions for use in a unifying container.

Some embodiments provide a kit including a syringe pre-filled with a composition including an naltrexone and pentosan polysulfate sodium, and instructions for use in a unifying container.

Some embodiments provide a kit including a syringe pre-filled with a composition including naltrexone and gabapentin, and instructions for use in a unifying container.

Some embodiments provide a kit including a syringe pre-filled with a composition including naltrexone, pentosan polysulfate sodium, and gabapentin, and instructions for use in a unifying container.

Some embodiments provide a kit including a first syringe pre-filled with a composition including an opioid antagonist and a second syringe pre-filled with a composition including a heparin-like compound, and instructions for use in a unifying container.

Some embodiments provide a kit including a first syringe pre-filled with a composition including an opioid antagonist and a second syringe pre-filled with gabapentin, and instructions for use in a unifying container.

Some embodiments provide a kit including a first syringe pre-filled with a composition including an opioid antagonist, a second syringe pre-filled with a heparin-like compound, and a third syringe pre-filled with gabapentin, and instructions for use in a unifying container.

Some embodiments provide a kit including a first syringe pre-filled with a composition including naltrexone and a second syringe pre-filled with pentosan polysulfate sodium, and instructions for use in a unifying container.

Some embodiments provide a kit including a first syringe pre-filled with a composition including naltrexone and a second syringe pre-filled with gabapentin, and instructions for use in a unifying container.

Some embodiments provide a kit including a first syringe pre-filled with a composition including naltrexone, a second syringe pre-filled with pentosan polysulfate sodium, and a third syringe pre-filled with gabapentin, and instructions for use in a unifying container.

Other embodiments provide kits including a vial or vials, or other appropriate container(s) containing a composition described herein and instruction for use in a unifying container. Still other embodiments provide a kit including a first and second vial or other appropriate container containing a first composition and a second composition and instruction for use in a unifying container. Still other embodiments provide a kit including a first, second, and third vial or other appropriate container(s) containing a first, second, and third compositions, and instruction for use in a unifying container.

Still other embodiments provide a kit including a vial or other appropriate container containing a composition described herein, a syringe, and instruction for use in a unifying container. Other embodiments provide a kit including a first and second vial or other appropriate container containing a first composition and a second composition, two syringes, and instruction for use in a unifying container. Other embodiments provide a kit including a first, second, and third vials or other appropriate container(s) containing a first, second, and third compositions, three syringes, and instruction for use in a unifying container.

Some embodiments provide a kit including any number of syringes pre-filled with a composition, two compositions, or three compositions as described herein and instruction for a scheduled use in a unifying container.

Other embodiments provide a kit including a vial(s) or other appropriate container containing a composition described herein for a scheduled use and instruction for use in a unifying container.

Once housed in a delivery device or included in a kit, the composition/delivery devices/kits can be sterilized using conventional sterilization techniques without substantial degradation to the composition. Without substantial degradation to the composition means that the composition retains greater than 80%, greater than 90%, greater than 95%, or greater than 99% of its activity. In some embodiments, the compositions remain unaffected by sterilization. Sterilization can be by at least one sterilization technique including, but not limited to gamma irradiation, pressure sterilization and/or steam sterilization.

In some embodiments, the compositions described herein can retain substantially all potency for at least 14 days. In some embodiments, the compositions described herein can retain substantially all potency for at least 30 days. In some embodiments, the compositions described herein can retain substantially all potency for at least 60 days. In some embodiments, the compositions described herein can retain substantially all potency for at least 90 days. In some embodiments, the compositions described herein can retain substantially all potency for at least 180 days. In some embodiments, the compositions described herein can retain substantially all potency for at least 360 days. In some embodiments, the compositions described herein can retain substantially all potency for longer than 360 days.

Substantially all potency means that the composition(s) retains at least greater than 80%, greater than 90%, greater than 95%, or greater than 99% of its activity when stored at appropriate conditions. In some embodiments, appropriate conditions can be a room temperature. In some embodiments, appropriate conditions can be without direct light. In some embodiments, appropriate conditions can be refrigerated.

The compositions described herein can reduce the incidence symptoms of pain.

In one embodiment, the compositions described herein can reduce the incidence symptoms of pain related to interstitial cystitis.

In one embodiment, the compositions described herein can reduce the incidence symptoms of acute pain.

In one embodiment, the compositions described herein can reduce the incidence symptoms of chronic pain.

In one embodiment, the compositions described herein can reduce the incidence symptoms of neuropathic pain.

In one embodiment, the compositions described herein can reduce the incidence symptoms of postoperative pain.

In one embodiment, the compositions described herein can reduce the incidence symptoms of bladder pain.

In one embodiment, the compositions described herein can reduce the incidence symptoms of muscle pain.

In one embodiment, the compositions described herein can reduce the incidence symptoms of wound pain.

In one embodiment, the compositions described herein can reduce the incidence symptoms of ligament pain.

In one embodiment, the compositions described herein can reduce the incidence symptoms of joint pain.

In one embodiment, the compositions described herein can reduce the incidence symptoms of inflammatory pain.

In one embodiment, the compositions described herein can reduce the incidence symptoms of surgical pain.

In one embodiment, the compositions described herein can reduce the incidence symptoms of pain associated with fibromyalgia.

In one embodiment, the compositions described herein can reduce the incidence symptoms of pain associated with Crohn's disease.

In one embodiment, the compositions described herein can reduce the incidence symptoms of pain associated with multiple sclerosis.

In some embodiments, the herein described compositions can reduce pain symptoms when compared to treatment with an opioid antagonist alone by at least about 10%, by at least about 20%, by at least about 50%, by at least about 75%, by at least about 100%, by at least about 150%, by at least about 200%.

In some embodiments, the herein described compositions can reduce joint disorder symptoms when compared to treatment with a heparin-like compound alone by at least about 10%, by at least about 20%, by at least about 50%, by at least about 75%, by at least about 100%, by at least about 150%, by at least about 200%.

In some embodiments, the herein described compositions can reduce joint disorder symptoms when compared to treatment with gabapentin alone by at least about 10%, by at least about 20%, by at least about 50%, by at least about 75%, by at least about 100%, by at least about 150%, by at least about 200%.

In some embodiments, the herein described compositions can reduce joint disorder symptoms when compared to treatment with naltrexone alone by at least about 10%, by at least about 20%, by at least about 50%, by at least about 75%, by at least about 100%, by at least about 150%, by at least about 200%.

In some embodiments, the herein described compositions can reduce joint disorder symptoms when compared to treatment with pentosan polysulfate sodium alone by at least about 10%, by at least about 20%, by at least about 50%, by at least about 75%, by at least about 100%, by at least about 150%, by at least about 200%.

In one embodiment, as a result of the reduced symptoms associated with pain as described herein, a composition(s) disclosed herein may reduce/remove/alleviate an unwanted side effect elicited by administration of an opioid antagonist and/or a heparin-like compound and/or gabapentin. In other embodiments, as a result of the reduced symptoms associated with pain as described herein, a composition disclosed herein may reduce an unwanted side effect elicited by administration of naltrexone and/or gabapentin and/or pentosan polysulfate sodium.

In one embodiment, unwanted side effects can include without limitation diarrhea, heartburn, stomach pain, hair loss, headache, rash, insomnia, bruising more easily, excess bleeding, constipation, injection site pain, mild rectal bleeding, mild rectal itching or discomfort, dizziness, chills, cold sweat, or pain during sexual intercourse.

Further, as a result of the synergistic and/or additive effects of the herein described compositions, a reduced load of drugs may be required to achieve similar results to the drugs alone. In some embodiments, treatment using the described compositions may only require about 10%, about 20%, about 30%, about 40%, about 50%, about 60%, about 70%, or about 80% of a general dose of an opioid antagonist, such as, but not limited to naltrexone. In other embodiments, treatment using the described compositions may only require about 10%, about 20%, about 30%, about 40%, about 50%, about 60%, about 70%, or about 80% of a general dose of gabapentin. In other embodiments, treatment using the described compositions may only require about 10%, about 20%, about 30%, about 40%, about 50%, about 60%, about 70%, or about 80% of a general dose of pentosan polysulfate sodium.

In some embodiments, compositions described herein can be administered regularly to aid in pain symptoms before they occur. The compositions can be administered to prevent recurrence of the pain symptoms. In other embodiments, the compositions can be administered before evidence of symptoms to prevent them.

### EXAMPLE 1

### Interstitial Cystitis Treatment

A patient having interstitial cystitis is treated with a combination of naltrexone and pentosan polysulfate sodium. The patient has extreme pain. The pentosan polysulfate sodium composition is injected directly into the bladder. The patient ingests orally, a low dose of naltrexone. The patient presents a reduction in pain and does not have typical side effects of high doses of naltrexone. Further the patient does not become addicted to naltrexone.

### EXAMPLE 2

### Knee Treatment

A patient having a knee injury is treated with a composition including naltrexone and gabapentin. The patient has extreme pain. The composition is injected locally into the knee. The patient presents a reduction in pain. The patient is given injections once a week to reduce the pain.

### EXAMPLE 3

### Hip Treatment

A patient having arthritis in her hip is treated with a composition including naltrexone and pentosan polysulfate sodium. The patient has extreme pain. The composition is injected locally into the hip. The patient presents a reduction in pain. The patient is given injections once a week to reduce the pain.

### EXAMPLE 4

### Pain Treatment

A patient having arthritis is treated with a composition including naltrexone, gabapentin, and pentosan polysulfate sodium. The patient has extreme pain. The composition is administered orally. The patient presents a reduction in pain. The patient is given oral doses once daily to reduce the pain.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A composition including:
an opioid antagonist, and
a heparin-like compound,
wherein the composition is formulated as a liquid for administration.

2. The composition of claim 1, wherein the opioid antagonist is naltrexone.

3. The composition of claim 1, wherein the opioid antagonist is present at about 0.1 mg to about 10 mg.

4. The composition of claim 1, wherein the heparin-like compound is pentosan polysulfate sodium.

5. The composition of claim 1, wherein the heparin-like compound is present at about 100 mg to about 300 mg.

6. The composition of claim 1, wherein the composition further includes gabapentin.

7. The composition of claim 1 formulated as a liquid.

8. A composition including:
an opioid antagonist, and
gabapentin,
wherein the composition is formulated as a liquid for administration.

9. The composition of claim 1, wherein the opioid antagonist is naltrexone.

10. The composition of claim 1, wherein the opioid antagonist is present at about 0.1 mg to about 10 mg.

11. The composition of claim 1, wherein the gabapentin is present at about 900 mg to about 1200 mg.

12. The composition of claim 1, wherein the composition further includes pentosan polysulfate sodium.

13. The composition of claim 1 formulated as a liquid.

14. A method of treating pain, the method comprising:
administering a composition including an opioid antagonist and a heparin-like compound to a mammal, and
treating the pain.

15. The method of claim 12, wherein the opioid antagonist is naltrexone.

16. The method of claim 12, wherein the opioid antagonist is present at between 0.1 mg and about 10 mg.

17. The method of claim 12, wherein the heparin-like compound is pentosan polysulfate sodium.

18. The method of claim 12, wherein the heparin-like compound is present at about 100 mg to about 300 mg.

19. The method of claim 12, wherein the composition further includes gabapentin.

20. A method of treating pain, the method comprising:
administering a composition including an opioid antagonist and gabapentin to a mammal, and
treating the pain.

21. The method of claim 20, wherein the opioid antagonist is naltrexone.

22. The method of claim 20, wherein the opioid antagonist is present at between 0.1 mg and about 10 mg.

23. The method of claim 20, wherein the pentosan polysulfate sodium is present at about 100 mg to about 300 mg.

24. The method of claim 20, wherein the composition further includes gabapentin.
